(19) United States
(12) Patent Application Publication
(10) Pub. No.: US 2006/0121619 A1
(43) Pub. Date: Jun. 8, 2006

(54) Protein biomarkers and therapeutic targets in an animal model for amyotrophic lateral sclerosis

(75) Inventor: Robert P. Bowser, Cranberry Township, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: University of Pittsburgh of the Commonwealth System of Higher Education, Pittsburgh, PA (US)

(21) Appl. No.: 11/294,326

(22) Filed: Dec. 2, 2005

Related U.S. Application Data
(60) Provisional application No. 60/632,380, filed on Dec. 2, 2004.

(51) Int. Cl. G01N 33/00

(52) U.S. Cl. 436/86

(57) ABSTRACT

The invention provides a method for determining the onset and/or progression of ALS in an animal. The method comprises (a) obtaining a sample from the animal, (b) analyzing the proteins in the sample by mass spectroscopy, and (c) determining a mass spectral profile for the sample. The invention also provides isolated protein biomarkers of ALS.

### CROSS-REFERENCE TO RELATED APPLICATIONS

This patent application claims the benefit of U.S. Provisional Patent Application No. 60/632,380, filed Dec. 2, 2004.

### STATEMENT REGARDING FEDERALLY SPONSORED RESEARCH AND DEVELOPMENT

This invention was made in part with Government support under Grant Number ES013469 awarded by the National Institute of Environmental Health Sciences. The Government may have certain rights in this invention.

### BACKGROUND OF THE INVENTION

Amyotrophic lateral sclerosis (ALS), also known as Lou Gehrig's disease or motor neuron disease (MND), is one of several neurodegenerative diseases of the central nervous system. ALS is the most common adult onset motor neuron disease, affecting one in every 20,000 individuals, with an average age of onset of 50-55 years. ALS is characterized by rapidly progressive degeneration of motor neurons in the brain, brainstem, and spinal cord (Cleveland, 2001). The median survival of patients from time of diagnosis is five years.

ALS exists in both sporadic and familial forms. Familial ALS (FALS) comprises only 5-10% of all ALS cases. Over the last decade, a number of basic and clinical research studies have focused on understanding the familial form of the disease, which has led to the identification of eight genetic mutations related to FALS. Transgenic mice expressing point mutants of the Cu/Zn superoxide dismutase-1 (SOD1) gene develop an age-dependent progressive motor weakness similar to human ALS due to a toxic gain of function (Rosen, 1993; Rosen, 1994; Borchelt, 1994).

These genetic mutations, however, do not explain sporadic ALS (SALS). The pathogenesis of SALS is multifactorial. A number of different model systems, including SOD1 transgenic mice, in vitro primary motor neuron cultures or spinal cord slice cultures, in vivo imaging studies, and postmortem examination of tissue samples, have been utilized to understand the pathogenesis of ALS (Subramaniam, 2002; Nagai, 2001; Menzies, 2002; Kim, 2003; Ranganathan, 2003). Although these studies have yielded therapeutic targets and several clinical trials, there are no drugs that delay disease onset or prolong long-term survival of ALS patients. Riluzole (Rilutek®, Aventis), a glutamate antagonist, currently is the only FDA-approved medication available to treat ALS. Riluzole, however, extends life expectancy by only a few months (Miller, 2003). Creatine and α-tocopherol have shown some efficacy in relieving the symptoms of ALS in SOD1 transgenic mice, but exhibit minimal efficacy in human ALS patients (Groeneveld, 2003; Desnuelle, 2001).

Studies have been performed which have identified early protein biomarkers for ALS, using mass spectrometry based proteomics of cerebrospinal fluid (CSF) and spinal cord samples of human subjects (see U.S. patent application Ser. No. 10/972,732, the disclosure of which is incorporated herein). There remains a need, however, for improved methods for identifying therapeutic targets of ALS, and improved methods of diagnosing and monitoring the progress of the disease.

Protein biomarkers common between humans with ALS and animal models of motor neuron disease have been identified in this study. These common biomarkers provide insight into disease mechanisms common between humans and animal models of ALS and targets for therapeutic intervention. Therapies that target these biomarkers within the animal model and can successfully affect the biomarker and impede or inhibit disease progression should then be tried in human clinical trials. Biomarkers common between animal models of disease and ALS patients provide a strong indication that drugs effective in the animal model may be effective in humans with disease.

### BRIEF SUMMARY OF THE INVENTION

The invention provides a method for determining the onset of ALS in an animal. The method comprises (a) obtaining a sample from the animal, (b) analyzing the proteins in the sample by mass spectroscopy, (c) determining a mass spectral profile for the sample, and (d) comparing the mass spectral profile of the sample to the mass spectral profile of a sample obtained from an animal that does not suffer from ALS or motor neuron degeneration, wherein protein biomarkers of ALS or motor neuron degeneration are identified.

The invention also provides a method for determining the onset of ALS in an animal. The method comprises (a) obtaining a sample from the animal, (b) analyzing the proteins in the sample by mass spectroscopy, and (c) determining a mass spectral profile for the sample, where a mass spectral profile comprising one or more biomarkers selected from the group consisting of a 4369 Dalton (Da) protein peak, a 6840 Da protein peak, a 6865 Da protein peak, a 7006 Da protein peak, an 8132 Da protein peak, an 8220 or 8230 Da protein peak, an 8310 Da protein peak, an 8611 Da protein peak, an 8730 Da protein peak, an 8806 Da protein peak, and a 9076 Da protein peak indicates onset of ALS in the animal, and a mass spectral profile comprising a 12.2 kDa protein peak indicates that the animal does have ALS.

Further provided is a method for determining progression of ALS in an animal, which method comprises (a) obtaining a sample from the animal, (b) analyzing the proteins in the sample by mass spectroscopy, (c) determining a mass spectral profile for the sample, wherein a mass spectral profile comprising one or more biomarkers selected from the group consisting of a 4367 Da protein peak, a 4660 Da protein peak, an 8547 Da a protein peak, an 8611 Da protein peak, an 8725 Da protein peak, an 8735 Da protein peak, an 8737 Da protein peak, an 8943 Da protein peak, and a 9528 Da protein peak, and (d) comparing the mass spectral profile to a mass spectral profile obtained from the same animal at an earlier time, wherein the presence of one or more biomarkers or an increase in the peak intensity of one or more biomarkers in the later mass spectral profile indicates progression of ALS in the animal.

In addition, the invention provides a method for determining the onset of ALS in an animal. The method comprises (a) obtaining a sample from the animal, (b) analyzing the proteins in the sample by mass spectroscopy, and (c) determining a mass spectral profile for the sample, wherein a mass spectral profile comprising one or more biomarkers selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak indicates onset of ALS in the animal.

In addition, the invention provides a method for determining progression of ALS in an animal. The method comprises, consists of, or consists essentially of (a) obtaining a sample from the animal, (b) analyzing the proteins in the sample by mass spectroscopy, (c) determining a mass spectral profile for the sample, wherein the mass spectral profile comprises one or more biomarkers selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak, and (d) comparing the mass spectral profile to a mass spectral profile obtained from the same animal at an earlier time, wherein the presence of one or more biomarkers or an increase in the peak intensity of one or more biomarkers in the later mass spectral profile indicates progression of ALS in the animal.

The invention also provides an isolated protein biomarker of amyotrophic lateral sclerosis selected from the group consisting of a 4369 Da protein peak, a 6840 Da protein peak, a 6865 Da protein peak, a 7006 Da protein peak, an 8132 Da protein peak, an 8220 or 8230 Da protein peak, an 8310 Da protein peak, an 8611 Da protein peak, an 8730 Da protein peak, an 8806 Da protein peak, and a 9076 Da protein peak, a 12.2 kDa protein peak, and combinations thereof.

In addition, the invention provides an isolated protein biomarker of amyotrophic lateral sclerosis selected from the group consisting of a 4367 Da protein peak, a 4660 Da protein peak, an 8547 Da a protein peak, an 8611 Da protein peak, an 8725 Da protein peak, an 8735 Da protein peak, an 8737 Da protein peak, an 8943 Da protein peak, a 9528 Da protein peak, and combinations thereof.

The invention further provides an isolated protein biomarker of amyotrophic lateral sclerosis selected from the group consisting of a 2046 Da protein peak, a 3208 Da protein peak, a 4803 Da protein peak, a 5210 Da protein peak, a 5366 Da protein peak, a 6174 Da protein peak, a 6467 Da protein peak, a 7661 Da protein peak, a 8557 Da protein peak, a 9905 Da protein peak, a 10863 Da protein peak, a 12357 Da protein peak, a 14830 Da protein peak, a 14992 Da protein peak, a 15835 Da protein peak, a 16019 Da protein peak, a 16777 Da protein peak, and combinations thereof.

The invention also provides an isolated protein biomarker of amyotrophic lateral sclerosis selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak, wherein the peak is determined by mass spectroscopy.

### BRIEF DESCRIPTION OF THE SEVERAL VIEWS OF THE DRAWINGS

FIG. 1 presents data which identifies potential plasma protein biomarkers of mutant SOD1 mice. Protein peaks with statistically significant (p<0.01) differences between day 90 mutant SOD1 and non-transgenic control littermates were identified using mass spectroscopy. By comparing the spectra from 10 mutant SOD1 mice and 10 control littermates 6 m/z peaks with highly significant differences in relative peak intensity values were discovered. These peaks were identified using Ciphergen based BPS software and univariate analysis. Error bars were not included in the graph.

FIG. 2 presents data that identifies potential plasma protein biomarkers that distinguish day 120 mutant SOD1 mice from control mice. Protein peaks with statistically significant (p<0.01) differences between mutant SOD1 and non-transgenic control littermates were identified using mass spectroscopy. Comparing the spectra from 10 mutant SOD1 mice and 10 control littermates, 9 m/z peaks with highly significant differences in relative peak intensity values were discovered. These peaks were identified using Ciphergen based BPS software and univariate analysis. Error bars were not included in this graph. The 7006 Da, 8612 Da, and 12.2 kDa peaks were present in both the day 90 (FIG. 1) and day 120 (FIG. 2) experiments.

FIG. 3 depicts a SELDI-TOF-MS spectra for the 12.2 kDa peak of control and mutant SOD1 mice at various ages. The 12.2 kDa protein was present in the plasma of the control non-transgenic mice of all ages (row 1-2, day 90 and Day 120 control mice, respectively). The 12.2 kDa protein was present at low levels of mutant SOD1 mice at day 50 (row 3) and absent in mutant SOD1 mice at days 90, 105, and 120 (rows 4-6). While the day 50 spectra in this figure fails to exhibit the 12.2 kDa peak, most other day 50 mutant SOD1 mice exhibit at least 50% the level of this peak observed in the control mice, suggesting that the level of this protein decreases as the mutant mice age.

FIG. 4 depicts a classification tree analysis for predictive biomarkers using Biomarker Patterns Software (BPS). BPS software easily distinguished Day 90 G93A mutant SOD1 expressing mice from Day 90 wildtype SOD1 expressing mice using the peak intensity value of the 12.2 kDa peak. Using 3-fold cross validation of 40 samples the sensitivity was 95% and the specificity was 95%.

FIG. 5 presents data which identifies protein biomarkers for disease progression. SELDI-TOF-MS spectra of the plasma of mutant SOD1 day 90 and day 105 mice were compared. Five m/z peaks were observed with statistically significant (p<0.01) differences in relative peak intensity values. These peaks were identified using Ciphergen based BPS software and univariate analysis. Error bars were not included in the graph.

FIG. 6 presents data which identifies protein peaks with statistically significant (p<0.01) differences between mutant SOD1 day 90 and day 120 mice. SELDI-TOF-MS spectra of the mutant SOD1 day 90 and 120 were compared. 6 m/z peaks with highly significant differences in relative peak intensity values were identified using Ciphergen based BPS software and univariate analysis. Error bars were not included in the graph. The 8547 Da, 8611 Da, and 8735 Da peaks were present in both the day 90/day 105 experiment (FIG. 5) and the day 90/day 120 experiment (FIG. 6).

FIG. 7 depicts representative spectra from Day 60, 90 and 120 G93A mutant SOD1 mice. The overall spectra are quite similar between Day 60 and Day 90, while peak differences can be observed near the end-stage of the disease at Day 120.

FIG. 8 presents data which identifies protein peaks with statistically significant (p<0.01) differences among mutant SOD1 mice at various ages during disease progression. By comparing spinal cord tissue spectra from 10 mutant SOD1 mice of various ages during disease progression, 18 m/z peaks with highly significant differences in relative peak intensity values were identified. These peaks were identified using Ciphergen based BPS software and univariate analysis. Error bars were not included in the graph. Some peaks increased in intensity during disease progression while others either decreased or exhibited an initial increase in peak intensity followed by decreased levels at end-stage (Day 120).

FIG. 9 depicts representative spectra comparing ion exchange fractionation of murine plasma samples 120 day old wild type control and transgenic mutant G93A SOD1 mice.

FIG. 10 presents data which identifies protein peaks with significant differences among mutant SOD1 transgenic mice and controls. The peaks were identified by analyzing the ion exchange fractionation samples using mass spectroscopy.

### DETAILED DESCRIPTION OF THE INVENTION

The present invention provides a method for determining the onset of ALS in an animal. The method comprises, consists of, or consists essentially of (a) obtaining a sample from the animal, (b) analyzing the proteins in the sample by mass spectroscopy, (c) determining a mass spectral profile for the sample, and (d) comparing the mass spectral profile of the sample to the mass spectral profile of a sample obtained from an animal that does not suffer from ALS or motor neuron degeneration, wherein protein biomarkers of ALS or motor neuron degeneration are identified.

The animal can be any suitable animal, but preferably is a mammal, such as a mouse, rat, monkey, or human. It is contemplated that the aforementioned inventive method can be used to diagnose ALS in animal models of the disease, in which case the subject is a non-human animal (e.g., a mouse, rat, monkey, dog, etc.). In a preferred embodiment, the subject is a human.

The term “sample”, as used herein refers to biological material isolated from an animal. The sample can contain any suitable biological material, but preferably comprises cells obtained from a particular tissue or biological fluid. The sample can be isolated from any suitable tissue or biological fluid. In this respect, the sample can be blood, blood serum, plasma, urine, or spinal cord tissue. In that ALS affects the central nervous system, the sample preferably is isolated from tissue or biological fluid of the central nervous system (CNS) (i.e., brain and spinal cord). In a preferred embodiment of the invention, the sample is isolated from cerebrospinal fluid (CSF). In fact, CSF from ALS patients has been used for biochemical assays that have identified changes in the levels of glutamate, glutamine synthetase, transglutaminase activity, γ-aminobutyric acid, and various markers of oxidative injury (see, e.g., Spreux-Varoquaux, 2002; Shaw, 2000; Smith, 1998), the disclosures of which are incorporated herein by reference).

The sample can be obtained in any suitable manner known in the art, such as, for example, by biopsy, blood sampling, urine sampling, lumbar puncture (i.e., spinal tap), ventricular puncture, and cisternal puncture. In a preferred embodiment of the invention, the sample is obtained by lumbar puncture, which also is referred to as a spinal tap or CSF collection. Lumbar puncture involves insertion of a spinal needle, usually between the 3rd and 4th lumbar vertebrae, into the subarachnoid space where CSF is collected. In instances where there is lumbar deformity or infection which would make lumbar puncture impossible or unreliable, the sample can be collected by ventricular puncture or cisternal puncture. Ventricular puncture typically is performed in human subjects with possible impending brain herniation. Ventricular puncture involves drilling a hole in the skull and inserting a needle directly into the lateral ventricle of the brain to collect CSF. Cisternal puncture involves insertion of a needle below the occipital bone (back of the skull), and can be hazardous due to the proximity of the needle to the brain stem. Many neurodegenerative diseases, such as Alzheimer's disease, Parkinson's disease, Huntington's disease, and ALS are characterized by the accumulation or presence of protein abnormalities which contribute to the disease phenotype, and are thus sometimes referred to in the art as “proteinopathies” (Jellinger, 1999; Paulson, 1999). The collection of all of the proteins and peptide sequences present within a biological sample at a given time often is referred to in the art as the “proteome.” Thus, the inventive method provides a means to analyze the proteome of a particular sample. One of ordinary skill in the art will appreciate that a proteomic analysis of the proteins present in a biological sample involves the systematic separation, identification, and characterization of all peptide sequences within the sample. The proteins in the sample can be separated by any suitable method known in the art. Suitable methods include, for example, centrifugation, ion exchange chromatography, reversed-phase liquid chromatography, and gel electrophoresis. Preferably, the proteins in the sample are separated using gel electrophoresis (e.g., one-dimensional or two-dimensional gel electrophoresis). Most preferably, the proteins in the sample are separated by subjecting the sample to two-dimensional gel electrophoresis (2DGE). 2DGE typically involves separation of proteins in a first dimension by charge using isoelectric focusing (IEF). The charge-focused proteins are then separated in a second dimension according to size by using an SDS-polyacrylamide gel (see, e.g., Lin, 2003; Ong, 2001, the disclosures of which are herein incorporated by reference).

Following separation of the proteins in the sample, each of the proteins can be isolated from the separation medium. The proteins can be isolated using any suitable technique, such as by extracting the protein “spots” from the gel. Extraction of protein spots from a gel typically involves the physical cutting of the spot from the gel.

Once the proteins in the sample are separated, the inventive method comprises analyzing the proteins in the sample by mass spectroscopy. In mass spectroscopy, a substance is bombarded with an electron beam having sufficient energy to fragment the molecule. The positive fragments that are produced (cations and radical cations) are accelerated in a vacuum through a magnetic field and are sorted on the basis of mass-to-charge ratio (m/z). Since the bulk of the ions produced in the mass spectrometer carry a unit positive charge, the value m/z typically is equivalent to the molecular weight of the fragment. Any suitable mass spectroscopy method can be used in connection with the inventive method. Examples of suitable mass spectroscopy methods include matrix-assisted laser desorption/ionization mass spectroscopy (MALDI), matrix-assisted laser desorption/ionization-time of flight (MALDI-TOF) mass spectroscopy, plasma desorption/ionization mass spectroscopy (PDI), electrospray ionization mass spectroscopy (ESI), and surface enhanced laser desorption/ionization-time of flight (SELDI-TOF) mass spectroscopy. In time-of-flight (TOF) methods of mass spectroscopy, charged (ionized) molecules are produced in a vacuum and accelerated by an electric field produced by an ion-optic assembly into a free-flight tube or drift time. The velocity to which the molecules may be accelerated is proportional to the square root of the accelerating potential, the square root of the charge of the molecule, and inversely proportional to the square root of the mass of the molecule. The charged molecules travel down the TOF tube to a detector. Mass spectroscopy methods which can be adapted for use in the inventive method are further described in, for example, International Patent Application Publication No. WO 93/24834, U.S. Pat. No. 5,792,664, U.S. Patent Application Publication No. 2004/0033530 A1, and Hillenkamp, 1990, the disclosures of which are herein incorporated by reference.

In a preferred embodiment of the invention, the proteins in the sample are analyzed by SELDI-TOF mass spectroscopy. Surface enhanced desorption/ionization processes refer to those processes in which the substrate on which the sample is presented to the energy source plays an active role in the desorption/ionization process. In this respect, the substrate (e.g., a probe) is not merely a passive stage for sample presentation. Several types of surface enhanced substrates can be employed in a surface enhanced desorption/ionization process. In one embodiment, the surface comprises an affinity material, such as anion exchange groups or hydrophilic groups (e.g., silicon oxide), which preferentially bind certain classes of molecules. Examples of such affinity materials include, for example, silanol (hydrophilic), C₈ or C₁₆ alkyl (hydrophobic), immobilized metal chelate (coordinate covalent), anion or cation exchangers (ionic) or antibodies (biospecific). The sample is exposed to a substrate bound adsorbent so as to bind analyte molecules according to the particular basis of attraction. When the analytes are biomolecules (e.g., proteins), an energy absorbing material (e.g., matrix) typically is associated with the bound sample. A laser is then used to desorb and ionize the analytes, which are detected with a detector. For SELDI-TOF mass spectroscopy, the mass accuracy for each protein peak is +/− 0.2%. SELDI-TOF mass spectroscopy systems are commercially available from, for example, Ciphergen Biosystems, Inc. (Fremont, Calif.). Surface enhanced desorption/ionization methods are described in, e.g., U.S. Pat. Nos. 5,719,060, 6,294,790, and 6,675,104, and International Patent Application Publication No. WO 98/59360, the disclosures of which are herein incorporated by reference.

One of ordinary skill in the art will appreciate that the output of a mass spectroscopy analysis is a plot of relative intensity as a function of the mass-to-charge ratio (m/z) of the proteins in the sample, which is referred to as a “mass spectral profile” or “mass spectrum.” The mass spectral profile, which typically is represented as a histogram depicting protein “peaks,” serves to establish the molecular weight and structure of the compound being analyzed. Thus, the inventive method further comprises determining a mass spectral profile for the sample. The most intense peak in the spectrum is termed the base peak, and all other peaks are reported relative to the intensity of the base peak. The peaks themselves typically are very sharp, and are often simply represented as vertical lines.

The ions that are formed by fragmentation of the proteins in the sample during mass spectroscopy are the most stable cations and radical cations formed by the protein molecules. The highest molecular weight peak observed in a spectrum typically represents the parent molecule less an electron, and is termed the molecular ion (M+). Generally, small peaks are also observed above the calculated molecular weight due to the natural isotopic abundance of ¹³C, ²H, etc. Many molecules with especially labile protons do not display molecular ions. For example, the highest molecular weight peak in the mass spectrum of alcohols occurs at an m/z one less than the molecular ion (m−1). Fragments can be identified by their mass-to-charge ratio, but it is often more informative to identify them by the mass which has been lost. For example, loss of a methyl group will generate a peak at m−15, while loss of an ethyl will generate a peak at m−29.

The inventive method further comprises, consists of, or consists essentially of comparing the mass spectral profile of the sample to the mass spectral profile of a sample obtained from an animal that does not suffer from ALS or motor neuron degeneration. Upon comparison of the samples, molecules that are indicators of a particular disease, or disease progression can be identified. Such indicator molecules also are referred to as “biomarkers,” and typically are proteins or protein fragments.

The mass spectral profile of the sample from the animal suffering from a particular disease can comprise, consist of, or consist essentially of any suitable number of biomarkers. The mass spectral profile of the animal suffering from ALS or a motor neuron degeneration typically comprises, consists of, or consists essentially of one or more biomarkers selected from the group consisting of a 4369 Dalton (Da) protein peak, a 6840 Da protein peak, a 6865 Da protein peak, a 7006 Da protein peak, an 8132 Da protein peak, an 8220 or 8230 Da protein peak, an 8310 Da protein peak, an 8611 Da protein peak, an 8730 Da protein peak, an 8806 Da protein peak, a 9076 Da protein peak, and a 12.2 kDa protein peak. The presence of the aforementioned biomarkers can be associated with abnormalities in protein expression levels (e.g., as a result of protein overexpression), abnormal proteolytic processing, and abnormal post-translational modification of proteins (e.g., glycosylation or oxidation).

In a preferred embodiment, the present invention provides a method for determining the onset of ALS in an animal. The method comprises, consists of, or consists essentially of (a) obtaining a sample from the animal, (b) analyzing the proteins in the sample by mass spectroscopy, and (c) determining a mass spectral profile for the sample. The inventive method is performed substantially as described above. The onset of ALS or a motor degenerative disease can be characterized, for example, by twitching, cramping, or stiffness of the muscles; muscle weakness affecting a leg; or difficulty chewing or swallowing. The mass spectral profile for determining the onset of ALS or a motor neuron degenerative disease in an animal preferably comprises, consists of, or consists essentially of one or more biomarkers selected from the group consisting of a 4369 Dalton (Da) protein peak, a 6840 Da protein peak, a 6865 Da protein peak, a 7006 Da protein peak, an 8132 Da protein peak, an 8220 or 8230 Da protein peak, an 8310 Da protein peak, an 8611 Da protein peak, an 8730 Da protein peak, an 8806 Da protein peak, a 9076 Da protein peak, and a 12.2 kDa protein peak.

Thus, in accordance with the inventive method, the onset of ALS occurs when the mass spectral profile of a sample obtained from an animal of interest (e.g., a human) comprises, consists of, or consists essentially of one or any combination of the biomarkers observed in the mass spectral profile of an animal suffering from ALS or motor neuron degeneration as described above. In this respect, the onset of ALS occurs when the mass spectral profile of a sample obtained from an animal comprises, consists of, or consists essentially of one or more, two or more, three or more, four or more, or even five or more (e.g., 6, 7, 8, 9, 10, or 15) of the protein peaks distinct for an animal suffering from ALS or motor neuron degeneration. Indeed, particular subsets of the protein peaks set forth above can have diagnostic significance with respect to ALS, such as for example a 7006 Da protein peak, a 8611 Da protein peak, and a 12713 Da protein peak. In addition, the 12.2 kDa protein peak alone can, for example, indicate that the animal has developed ALS. These subsets, however, are merely exemplary, and any suitable subset of the biomarkers identified herein can be used to determine the onset of ALS. In addition, the onset of ALS can be confirmed by comparing the mass spectral profile of the sample to the mass spectral profile of an animal that does not suffer from ALS or motor neuron degeneration. In this respect onset is confirmed when the mass spectral profile of the animal that does not suffer from ALS does not comprise, consist of, or consist essentially of any of the biomarkers observed in the mass spectral profile of the sample. In addition, a determination of ALS onset can be made if a sample obtained from an animal comprises, consists of, or consists essentially of one or more fragments or full-length amino acid sequences of the biomarkers in the mass spectral profile of an animal suffering from ALS or a motor degenerative disease. Moreover, as a result of post-translational modification of proteins, it is also contemplated that determination of the site of ALS onset can be made when the sample obtained from an animal comprises, consists of, or consists essentially of a modified form (e.g., a glycosylated form) of one or more of the biomarkers.

Further provided is a method for determining progression of ALS in an animal, which method comprises (a) obtaining a sample from the animal, (b) analyzing the proteins in the sample by mass spectroscopy, (c) determining a mass spectral profile for the sample, wherein the mass spectral profile comprises one or more biomarkers selected from the group consisting of a 4367 Da protein peak, a 4660 Da protein peak, an 8547 Da a protein peak, an 8611 Da protein peak, an 8725 Da protein peak, an 8735 Da protein peak, an 8737 Da protein peak, an 8943 Da protein peak, and a 9528 Da protein peak, and (d) comparing the mass spectral profile to a mass spectral profile obtained from the same animal at an earlier time, wherein the presence of one or more biomarkers or an increase in the peak intensity of one or more biomarkers in the later mass spectral profile indicates progression of ALS in the animal.

The mass spectral profile described above can be compared to any mass spectral profile obtained from the same animal at any point in time which is earlier than the time at which the mass spectral profile was obtained. As discussed above, the determination of the progression of ALS in an animal does not require that all of the above-described biomarkers be present in the sample obtained from the animal of interest. Indeed, a determination of the progression of ALS can be made if a sample obtained from the animal, when compared with a sample obtained from the same animal at an earlier time, comprises, consists of, or consists essentially of any additional biomarker, combination of, or subset of the above-described biomarkers (e.g., 4367 Da, 8547 Da, 8611 Da). In addition, a determination of the progression of ALS can be made if a sample obtained from the animal, when compared with a sample obtained from the same animal at an earlier time, comprises, consists of, or consists essentially of one or more additional fragments or full-length amino acid sequences of the biomarkers identified above. A determination of the progression of ALS can also be made if a sample obtained from the animal, when compared with a sample obtained from the same animal at an earlier time, comprises, consists of, or consists essentially of an increase in the size of any of the peaks of one or more of the identified biomarkers. Moreover, as a result of post-translational modification of proteins, it is also contemplated that determination of the progression of ALS can be made when the sample obtained from an animal, when compared with a sample obtained from the same animal at an earlier time, comprises, consists of, or consists essentially of a modified form (e.g., a glycosylated form) of at least one additional biomarker identified above.

In addition, the invention provides a method for determining the onset of ALS in an animal. The method comprises (a) obtaining a sample from the animal, (b) analyzing the proteins in the sample by mass spectroscopy, and (c) determining a mass spectral profile for the sample, wherein a mass spectral profile comprising one or more biomarkers selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak indicates onset of ALS in the animal. The method can be performed substantially as described above.

In addition, the invention provides a method for determining progression of ALS in an animal. The method comprises, consists of, or consists essentially of (a) obtaining a sample from the animal, (b) analyzing the proteins in the sample by mass spectroscopy, (c) determining a mass spectral profile for the sample, wherein the mass spectral profile comprises one or more biomarkers selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak, and (d) comparing the mass spectral profile to a mass spectral profile obtained from the same animal at an earlier time, wherein the presence of one or more biomarkers or an increase in the peak intensity of one or more biomarkers in the later mass spectral profile indicates progression of ALS in the animal. The method can be performed substantially as described above.

Using proteomic techniques, protein biomarkers common between humans with ALS and animal models of motor neuron disease can be identified. These common biomarkers provide insight into disease mechanisms common between humans and animal models of ALS and targets for therapeutic intervention. Therapies that target these biomarkers within the animal model and can successfully affect the biomarker and impede or inhibit disease progression should then be tried in human clinical trials. Biomarkers common between animal models of disease and ALS patients provide a strong indication that drugs effective in the animal model may be effective in humans with disease. For example, a series of mass spectral peaks common to a transgenic animal model of motor neuron disease and ALS patients is as follows:

TABLE 1

| | |
|---|---|
| Human Biomarker (kDa) | Mouse Biomarker (kDa) |

| CSF | Spinal Cord | Blood | Spinal Cord |
|---|---|---|---|
| 8.61 ↓ | | 8.61 ↑ | |
| 9.08 ↓ | | 9.08 ↓ | |
| 6.86 ↓ | | 6.86 ↓ | |
| 3.42 ↑ | 3.42 ↑ | | 3.42 ↑ |
| 4.80 ↑ | | | 4.80 ↑ |

These can similarly serve as biomarkers in the methods described above.

In another embodiment, the invention provides an isolated protein biomarker of amyotrophic lateral sclerosis selected from the group consisting of a 4369 Da protein peak, a 6840 Da protein peak, a 6865 Da protein peak, a 7006 Da protein peak, an 8132 Da protein peak, an 8220 or 8230 Da protein peak, an 8310 Da protein peak, an 8611 Da protein peak, an 8730 Da protein peak, an 8806 Da protein peak, a 9076 Da protein peak, a 12.2 kDa protein peak, and combinations thereof. The isolation of the biomarker can be performed substantially as described above.

In addition, the invention provides an isolated protein biomarker of amyotrophic lateral sclerosis selected from the group consisting of a 4367 Da protein peak, a 4660 Da protein peak, an 8547 Da a protein peak, an 8611 Da protein peak, an 8725 Da protein peak, an 8735 Da protein peak, an 8737 Da protein peak, an 8943 Da protein peak, a 9528 Da protein peak, and combinations thereof.

The invention further provides an isolated protein biomarker of amyotrophic lateral sclerosis selected from the group consisting of a 2046 Da protein peak, a 3208 Da protein peak, a 4803 Da protein peak, a 5210 Da protein peak, a 5366 Da protein peak, a 6174 Da protein peak, a 6467 Da protein peak, a 7661 Da protein peak, a 8557 Da protein peak, a 9905 Da protein peak, a 10863 Da protein peak, a 12357 Da protein peak, a 14830 Da protein peak, a 14992 Da protein peak, a 15835 Da protein peak, a 16019 Da protein peak, a 16777 Da protein peak, and combinations thereof. In a preferred embodiment, the protein biomarker is isolated from the spinal cord of the animal from which the sample is obtained.

The inventive biomarker can also be substantially purified from other proteins (e.g., at least about 90% pure or at least about 95% pure or even at least about 98% or 99% pure). Standard methods of protein purification (e.g., centrifugation, ion exchange chromatography, reversed-phase liquid chromatography, and gel electrophoresis) can be employed to substantially purify the protein.

The invention also provides an isolated protein biomarker of amyotrophic lateral sclerosis selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak, wherein the peak is determined by mass spectroscopy.

The inventive biomarker can also be substantially purified from other proteins (e.g., at least about 90% pure or at least about 95% pure or even at least about 98% or 99% pure). Standard methods of protein purification (e.g., centrifugation, ion exchange chromatography, reversed-phase liquid chromatography, and gel electrophoresis) can be employed to substantially purify the protein.

The following examples further illustrate the invention but, of course, should not be construed as in any way limiting its scope.

### EXAMPLES

Proteomic studies for ALS were performed in an animal model, which resulted in the discovery of biomarkers for disease onset and progression in the model. In addition, three of the protein peaks identified in the animal model were identified as having a mass peak identical to human ALS subjects. These protein peaks may highlight mechanisms in common in the animal model and ALS patients, and provide novel therapeutic targets.

Approximately 2-3% of ALS patients harbor mutations in the Cu/Zn SOD1 gene. Transgenic mice that over express mutant human Cu/Zn SOD1 have been produced that reproduce ALS pathogenesis. Transgenic mice that over express the G93A mutant SOD1 gene are the most commonly used animal model system for ALS, and have been used to identify disease mechanisms and for preclinical drug tests. The G93A SOD1 mice develop hindlimb weakness at approximately 90 days of age and rapidly progress till end-stage at approximately 120 days of age when they are sacrificed. Prior studies have attempted to identify early markers of disease onset prior to the clinical symptoms. These studies have demonstrated that inflammatory proteins increase within 50 days after birth of the transgenic mice. This animal model system has been utilized to discover protein biomarkers for disease onset and progression using mass spectrometry based proteomics with Ciphergen ProteinChips. This system uses charged surfaces on the ProteinChip to bind proteins and peptides from the biologic material that is then analyzed by mass spectrometry (SELDI-TOF-MS). G93A mutant SOD1 transgenic mice and control non-transgenic littermates at 50, 60, 90, 105, and 120 days of age were studied. Plasma and spinal cord tissues from 10 animals were obtained from the ALS Therapy Development Foundation in Boston, Mass. SELDI-TOF-MS was performed on plasma and lumbar spinal cord tissue homogenates from the G93A mutant SOD1 and non-transgenic littermate controls. Albumin was first eliminated from the plasma samples prior to analysis.

General Procedures

Transgenic Mice

The animal model used in this application is a transgenic mouse that over expresses the human mutant G93A Cu/Zn SOD1 gene. This animal is described in detail in Gurney, 1994. This animal model is typically used in preclinical studies to investigate potential drug interventions for ALS (Kriz, 2003; Rothstein, 2005; Gurney, 1996).

Sample Preparation

Blood plasma and spinal cords were obtained from transgenic mice expressing the human G93A Cu/Zn SOD1 gene and non-transgenic control littermates. These samples were obtained from the ALS Therapy Development Foundation (ALS-TDF) located in Boston, Mass. Spinal cord tissues were homogenized in lysis buffer containing 1% Triton X-100 in a polytron at 15,000 rpm for 45 seconds. Samples were centrifuged for 5 minutes at 3000 rpm and the supernatant removed and stored in a low-bind eppendorf tube at −80 C. until used for mass spectrometry.

Mass Spectroscopy

Mass spectrometry of plasma samples were performed either directly on the plasma samples or on each fraction obtained by ion exchange chromatography using SELDI ProteinChip® technology (Ciphergen Biosystems, Inc., Fremont, Calif.). Plasma samples were either directly analyzed on Q10 and Zinc coated IMAC30 Protein Chips, or first fractionated by ion exchange chromatography and then analyzed on gold Protein Chips. For analysis on the Ciphergen Gold Chips the following procedure was performed. A Gold chip was washed with water and rinsed with mild detergent (RBS 35). The chip is rinsed sequentially with water and methanol, and then placed on a heat block at 37° C. till dry. The matrix was prepared by adding approximately 1.0 mL of 0.1-0.5% TFA in 50% ACN to a small amount of sinapinic acid. The solution is vortexed and the mixture centrifuged to form a pellet. The supernatant is removed 5 μl of each ion exchange chromatography fraction is added to 5 μl of matrix (1:1 ratio). 2 μl of this protein/matrix mixture is spotted onto a spot on the Gold chip. After drying, an additional 2 μl of protein/matrix mixture is added to the well and once dry the Gold chips are analyzed by mass spectrometry. The resolution of the mass spectrometer is +/−10 Da and therefore each of the peaks identified has a mass error of +/−10 Da.

External calibration of the Protein Chip Reader was performed using the Ciphergen All-in-One peptide/protein standard mix containing peptides ranging from 1000 Da to 20 kDa. The dried chips were immediately loaded into the calibrated Chip Reader using optimal laser intensity and detector sensitivity with a mass deflector setting of 1000 Da for low mass range (2-20 kDa) and 10,000 Da for high mass range (20 kDa-80 kDa). These settings were kept constant for all the chips of every experiment. The mass/charge (m/z) ratios were determined using time of flight (TOF) analysis. These spectras were collected with a Protein Chip system (PBS II series; Ciphergen Biosystems Inc., Palo Alto).

Ion Exchange Chromatography

30 μl of 9M urea buffer was added to 20 μl of mouse plasma and mixed for 15 minutes at room temperature. 200 μl of 50 mM HEPES pH 9.0 was added and the mixture was shaken for 5 min. 75 μl of Q Ceramic HyperD F was spin at 3000 rpm for 1 minute and the supernatant was removed. 200 μl of pH 9.0 buffer was added, mixed for 1 minute, and spun at 3000 rpm for 30 seconds. The supernatant was removed and the procedure was repeated two more times. Add 500 μl of 1M urea buffer was added, mixed for 5 min, and spun at 3000 rpm for 30 sec. The supernatant was removed. The sample was added into the tube containing Q Ceramic HyperD F. The tubes were placed on the nutator (Clay ADAMS) and allowed to bind for 60 min at room temperature. Tubes were then spun at 300 rpm for 30 sec and the supernatant was removed, placed in a new tube, and labeled as unbound sample. 100 μl of pH 9.0 buffer was added, mixed for 10 min at room temperature, and spun at 3000 rpm for 30 sec. The supernatant was removed to a new tube. The process was repeated one more time and the supernatants were pooled together and labeled as the pH 9.0 fraction. The process was repeated using buffers have a pH of 7.0, 5.0, 4.0 and 3.0. 200 μl of organic buffer was added, mixed for 5 min, and spin at 3000 rpm for 30 sec. The supernatant was removed and added to a new tube and labeled as the organic fraction. The samples were stored at −80 C. For mass spectroscopy analysis, 10 μl of half saturated SPA was added to 5 μl of each fraction and mix on Micromix 5 (DPC) for 1 min. 1.5 μl was spotted onto the gold chips. After drying, 1 μl of 50% saturated SPA was spotted onto each well, and the chip was read on the mass spectrometer.

Data Analysis

Protein peaks were analyzed with the Ciphergen Biomarker Patterns Software (BPS) package version 3.1 licensed by Ciphergen Biosystems, and the Rules Learning (RL) Parameters algorithm. BPS is a classification algorithm defining pattern recognition approach and building classification trees. The Ciphergen 3.1 Biomarker Wizard application autodetected mass peaks by clustering and analyzed the output using non-parametric Mann-Whitney statistical analysis, which constituted the univariate analysis of the data. Peak labeling was performed using second-pass peak selection with a signal to noise ratio of 1.5. Each tree comprises a parent node and branch nodes or terminal nodes. There is a relative cost value associated with each of these trees. The algorithm also calculates values for sensitivity and specificity of the peaks. Sensitivity is defined as the ratio of the number of correctly classified disease cases to total number of disease cases. Specificity is defined as the ratio of the number of correctly classified control cases to total number of control cases. A short tree size defining a low cost value signifies better classification of the two data groups. This also signifies a higher sensitivity and specificity of the peaks to describe the ability of the classification trees to differentiate the ALS and control groups. The final tree size is determined using a cross validation method, in which the tree is built on a fraction of the data and then the remainder of the data is utilized to assess the tree error rate. This tree building process determines the spectra most valuable in terms of delineating the two sets of data (i.e., ALS vs. control).

The Rules Learning (RL) parameters algorithm was first used to learn rules for predicting mass spectra of complex organic molecules (see, e.g., Feigenbaum et al., Artificial Intelligence, 59, 233-240 (1993)) and views inductive learning as a knowledge-based problem solving activity that can be implemented in the heuristic search paradigm. RL primarily searches possible rules by successive specialization, guided by data in the training set and by prior knowledge about the data (e.g., clinical diagnosis or symptoms, subject medications) to define diagnostic biomarkers.

RL has been applied to numerous scientific, commercial, and medical data sets (see, e.g., Lee, 1996). The main method adopted by RL is hypothesis testing through generation of hypotheses and testing by evidence gathering. Several different kinds of statistics are employed during evidence gathering. These include an estimation of certainty factor (cf) for each rule, together with its positive predictive value and p-value. The RL program will generate predictive rules from two-thirds of the samples within the dataset, reiterate this rule generation phase three times to develop the best rule set, and then apply these rules to the remaining one-third of the samples to test the ability of the rules to make proper predictions.

### Example 1

This example demonstrates the identification of protein biomarkers associated with the onset of ALS.

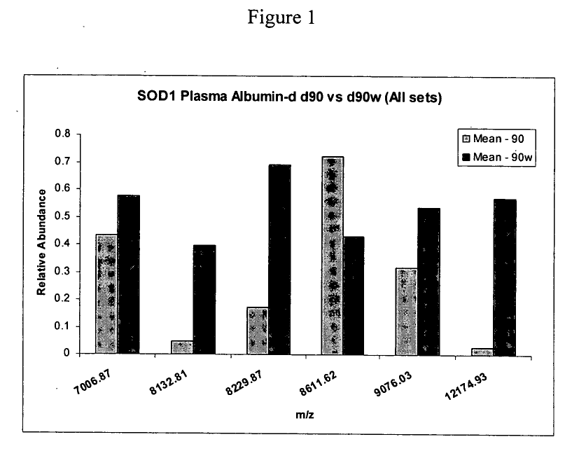

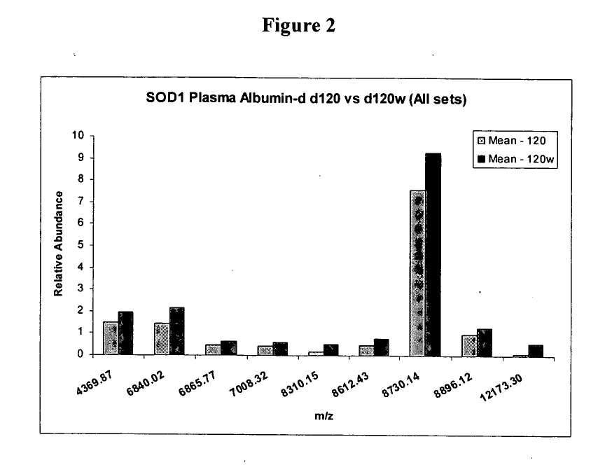

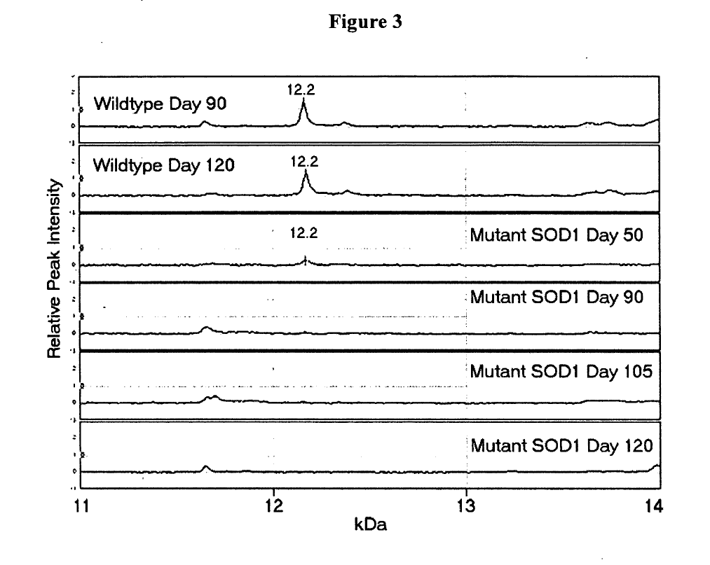

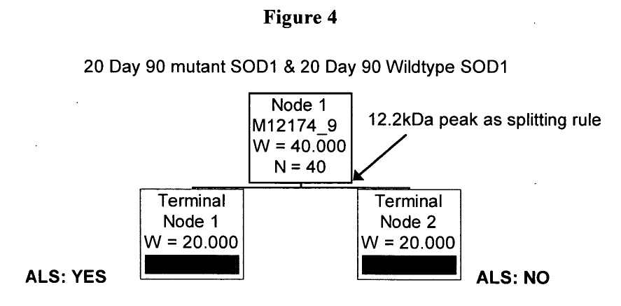

Differences in m/z peak intensity values were determined between Day 90 control and mutant SOD1 mice (FIG. 1). Peaks of 7006 Da, 8132 Da, 8220 Da, 8611 Da, 9076 Da and 12.2 kDa were detected that exhibit statistically significant differences in peak intensity values (p<0.01). These peaks likely represent protein biomarkers at the time of symptom onset in the animal model for ALS. By similarly comparing the spectra from Day 90 control and mutant SOD1 mice, nine putative biomarkers were uncovered (FIG. 2). These were 4369 Da, 6840 Da, 6865 Da, 7006 Da, 8310 Da, 8611 Da, 8730 Da, 8806 Da, and 12.2 kDa. Three peaks (7006 Da, 8611 Da, 12.2 kDa) were common between these two figures. These protein alterations may occur early in the disease pathogenesis and throughout the course of disease. It was also discovered that the 12.2 kDa peak was present in all control mice but was present in only some mutant SOD1 mice at 50 Days of age and absent in all mutant SOD1 transgenic mice Days 90, 105 and 120 (FIG. 3). Thus, a total of twelve putative biomarker peaks have been identified that can distinguish control from G93A mutant SOD1 transgenic mice. Analysis using a computer software classification tree algorithm was performed in order to determine particular peaks that could be used to distinguish mutant SOD1 mice from control littermates. Classification trees containing the 6840 Da and 12.2 kDa peaks could identify mutant SOD1 mice from control littermates with 95% sensitivity and 95% specificity (see FIG. 4).

### Example 2

This example demonstrates the identification of protein biomarkers associated with the progression of ALS.

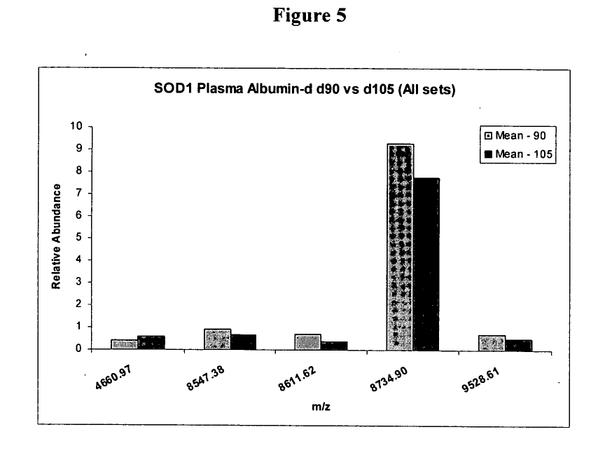

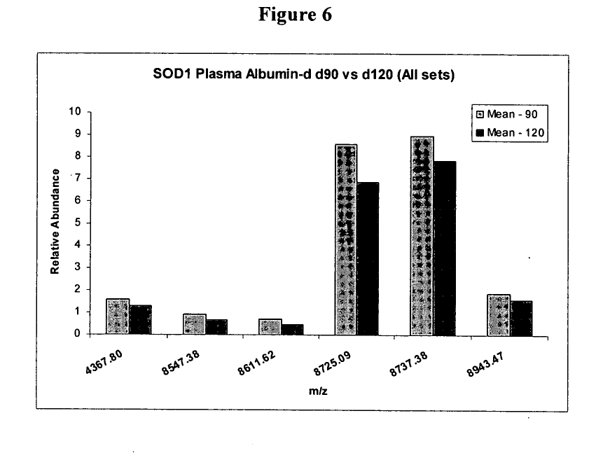

G93A mutant SOD1 transgenic mice were compared at various ages by SELDI-TOF-MS to uncover putative biomarkers for disease progression. By comparing spectra from Day 90 versus Day 105, five peaks at 4660 Da, 8547 Da, 8611 Da, 8735 Da, and 9528 Da were identified which exhibit statistically significant (p<0.01) differences in relative abundance (FIG. 5). Comparison of Day 90 to Day 120 mutant SOD1 mice uncovered six peaks of 4367 Da, 8547 Da, 8611 Da, 8725 Da, 8737 Da, and 8943 Da with statistically significant (p<0.01) differences in peak intensity values (FIG. 6). These protein peaks represent putative biomarkers for disease progression. Using classification tree algorithms, it was determined that a classification tree containing 4367 Da, 8547 Da, and 8611 Da could distinguish Day 90 from Day 120 mutant SOD1 mice with 95% sensitivity and 95% specificity.

### Example 3

This example demonstrates the identification of protein biomarkers within the spinal cord that are associated with ALS.

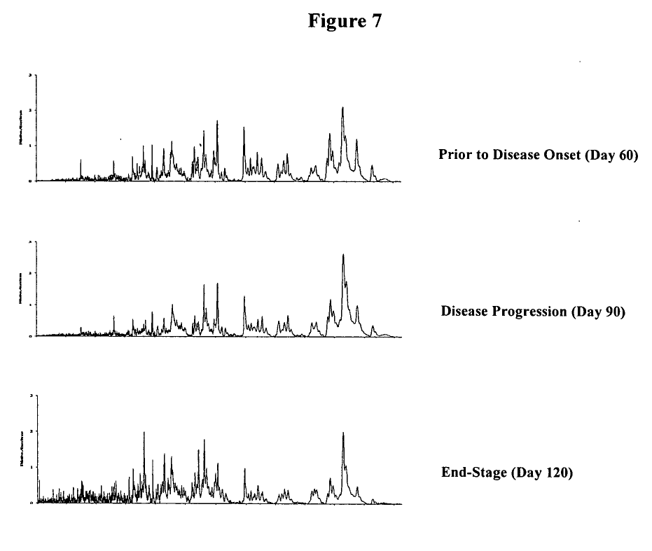

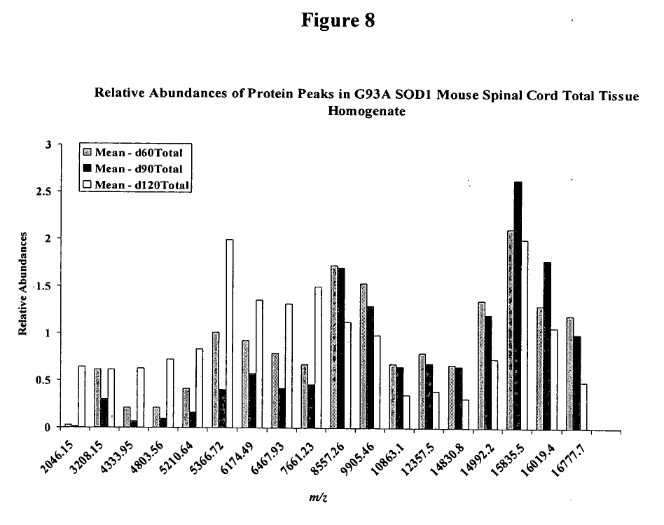

SELDI-TOF-MS analysis of lumbar spinal cord tissue samples was also performed to discover protein biomarkers within the spinal cord. Representative spectra for lumbar spinal cord tissue homogenates is shown in FIG. 7. Univariate comparison of the spectra for each age group revealed eighteen potential biomarkers that exhibit statistically significant (p<0.01) differences in peak intensity values between each age group (FIG. 8). These peaks are 2046 Da, 3208 Da, 4803 Da, 5210 Da, 5366 Da, 6174 Da, 6467 Da, 7661 Da, 8557 Da, 9905 Da, 10863 Da, 12357 Da, 14830 Da, 14992 Da, 15835 Da, 16019 Da, and 16777 Da. Four of these protein peaks, 4803 Da, 5366 Da, 8557 Da, and 16777 Da, have also been observed in the CSF of humans and are biomarkers for distinguishing the CSF of ALS from control subjects. Two additional m/z peaks of 6467 Da and 7661 Da also have similar m/z peaks in human CSF and exhibit statistically significant (p<0.01) differences between ALS and control subjects.

### Example 4

This example demonstrates the identification of protein biomarkers using ion exchange fractionation experiments.

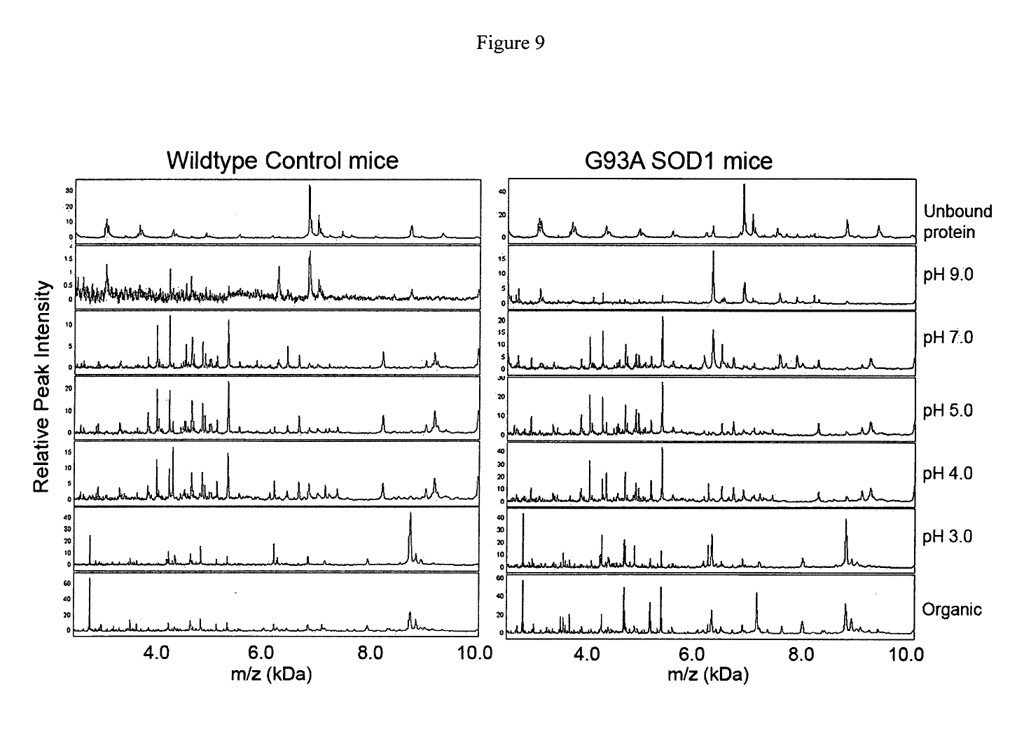

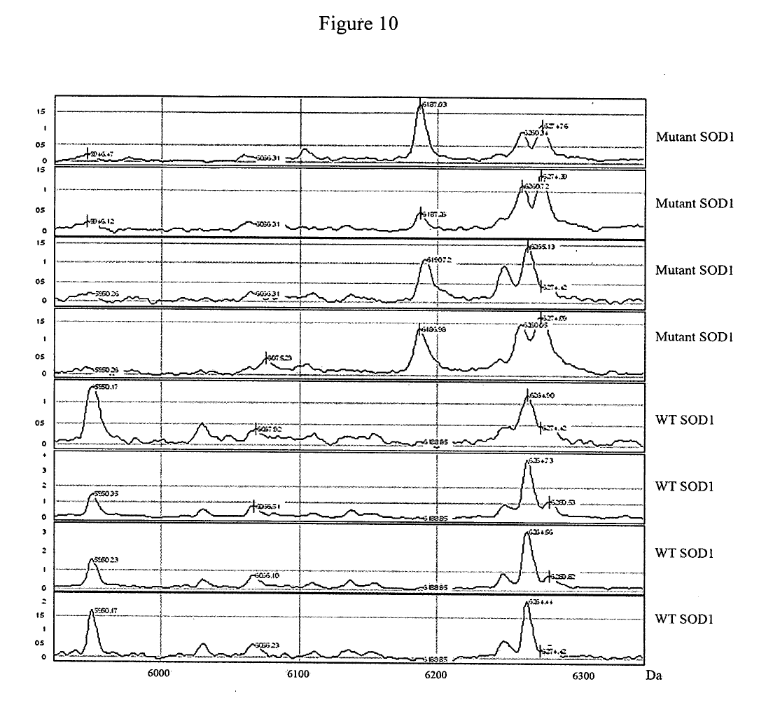

Ion exchange fractionation was performed on samples of mouse plasma substantially as described above. Samples were analyzed using a Ciphergen Gold Chips and chip reader. FIG. 9 shows data from 120 day old wild type control and transgenic mutant G93A SOD1 mice. The fractions analyzed consist of proteins that failed to bind to the column (unbound protein), proteins which eluted from the column at pH 9.0, 7.0, 5.0, 4.0, 3.0, and proteins that eluted from the column in the organic phase. The pH 3.0 fractions were further analyzed on gold chips by mass spectrometry. Mass peaks of 5960 Da, 6187 Da, 6260 Da, and 6274 Da were identified which differed between the control and transgenic G93A SOD1 mice (FIG. 10).

Thus, this invention utilizes an animal model of ALS to identify protein biomarkers for onset of disease and disease progression. In addition, particular m/z peaks have been found that are common to those in humans. These peaks highlight common biochemical pathways of pathogenesis common to humans and the animal model for ALS, and provide potential therapeutic targets.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Borchelt et al., Proc. Natl. Acad. Sci. USA, 91, 8292-8296 (1994).

Cleveland et al., Nat. Rev. Neurosci., 2, 806-19 (2001).

Desnuelle et al., Amyotrophic Lateral Sclerosis & Other Motor Neuron Disorders, 2, 9-18 (2001).

Groeneveld et al., Annals of Neurology, 53, 437-45 (2003).

Gurney et al., Science 264, 1772-1775 (1994).

Gurney et al., Ann. Neurol. 39, 147-157 (1996).

Hillenkamp et al., Matrix Assisted UV-Laser Desorption/Ionization: A New Approach to Mass Spectroscopy of Large Biomolecules, Biological Mass Spectroscopy, Burlingame and McCloskey, eds., Elsevier Science Publ., pp. 49-60 (1990).

Jellinger, Movement Disorders, 18, Suppl 6, S2-12 (2003).

Kim et al. J. Neuropathol. Exp. Neurol., 62, 88-103 (2003).

Kriz et al., Ann Neurol 53, 429-436 (2003).

Lee et al., Environ Health Perspect., 104 Suppl 5, 1059-63 (1996).

Lin et al., Biochimica et Biophysica Acta, 1646, 1-10 (2003).

Menzies et al. Brain, 125, 1522-1533 (2002).

Miller et al., Amyotrophic Lateral Sclerosis & Other Motor Neuron Disorders, 4, 191-206 (2003).

Nagai et al., J. Neurosci., 21, 9246-9254 (2001).

Ong et al., Biomol. Eng., 18, 195-205 (2001).

Paulson, American Journal of Human Genetics, 64, 339-45 (1999).

Ranganathan et al., Am. J. Pathol., 162, 823-835 (2003).

Ranganathan et al., J Neurochem, 9, 1461-1471 (2005).

Rosen et al., Nature, 362, 59-62 (1993).

Rosen et al., Hum Mol Genet, 3, 981-987 (1994).

Rothstein et al., Nature 403, 73-77 (2005).

Shaw et al., Amyotrophic Lateral Sclerosis & Other Motor Neuron Disorders, 1, Suppl. 2, S61-67 (2000).

Smith et al., Ann. Neurol., 44, 696-699 (1998).

Spreux-Varoquaux et al., Journal of the Neurological Sciences, 193, 73-78 (2002).

Subramaniam et al., Nat. Neurosci., 5, 301-307 (2002).

1. A method for identifying protein biomarkers of amyotrophic lateral sclerosis (ALS) or motor neuron degeneration in an animal suffering from ALS or motor neuron degeneration, which method comprises:
(a) obtaining a sample from the animal,
(b) analyzing the proteins in the sample by mass spectroscopy,
(c) determining a mass spectral profile for the sample, and
(d) comparing the mass spectral profile of the sample to the mass spectral profile of a sample obtained from an animal that does not suffer from ALS or motor neuron degeneration, wherein protein biomarkers of ALS or motor neuron degeneration are identified.

2. A method for determining the onset of ALS in an animal, which method comprises:
(a) obtaining a sample from the animal,
(b) analyzing the proteins in the sample by mass spectroscopy, and
(c) determining a mass spectral profile for the sample, wherein (i) a mass spectral profile comprising one or more biomarkers selected from the group consisting of a 4369 Dalton (Da) protein peak, a 6840 Da protein peak, a 6865 Da protein peak, a 7006 Da protein peak, an 8132 Da protein peak, an 8220 Da protein peak, an 8310 Da protein peak, an 8611 Da protein peak, an 8730 Da protein peak, an 8806 Da protein peak, and a 9076 Da protein peak indicates onset of ALS in the animal, and (ii) a mass spectral profile comprising a 12.2 kDa protein peak indicates that the animal does have ALS.

3. A method for determining progression of ALS in an animal, which method comprises:
(a) obtaining a sample from the animal,
(b) analyzing the proteins in the sample by mass spectroscopy,
(c) determining a mass spectral profile for the sample, wherein the mass spectral profile comprises one or more biomarkers selected from the group consisting of a 4367 Da protein peak, a 4660 Da protein peak, an 8547 Da a protein peak, an 8611 Da protein peak, an 8725 Da protein peak, an 8735 Da protein peak, an 8737 Da protein peak, an 8943 Da protein peak, and a 9528 Da protein peak, and
(d) comparing the mass spectral profile to a mass spectral profile obtained from the same animal at an earlier time, wherein the presence of one or more biomarkers or an increase in the peak intensity of one or more biomarkers in the later mass spectral profile indicates progression of ALS in the animal.

4. A method for determining the onset of ALS in an animal, which method comprises:
(a) obtaining a sample from the animal,
(b) analyzing the proteins in the sample by mass spectroscopy, and
(c) determining a mass spectral profile for the sample, wherein (i) a mass spectral profile comprising one or more biomarkers selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak indicates onset of ALS in the animal.

5. A method for determining progression of ALS in an animal, which method comprises:
(a) obtaining a sample from the animal,
(b) analyzing the proteins in the sample by mass spectroscopy,
(c) determining a mass spectral profile for the sample, wherein the mass spectral profile comprises one or more biomarkers selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak, and
(d) comparing the mass spectral profile to a mass spectral profile obtained from the same animal at an earlier time, wherein the presence of one or more biomarkers or an increase in the peak intensity of one or more biomarkers in the later mass spectral profile indicates progression of ALS in the animal.

6. The method of any of claims 1-5, wherein the animal is a mouse or rat.

7. The method of any of claims 1-5, wherein the animal is a human.

8. The method of any of claims 1-5, wherein the sample is selected from the group consisting of cerebrospinal fluid, blood serum, plasma, urine, and tissue obtained from the animal.

9. An isolated protein biomarker of amyotrophic lateral sclerosis selected from the group consisting of a 4369 Da protein peak, a 6840 Da protein peak, a 6865 Da protein peak, a 7006 Da protein peak, an 8132 Da protein peak, an 8220 Da protein peak, an 8310 Da protein peak, an 8611 Da protein peak, an 8730 Da protein peak, an 8806 Da protein peak, a 9076 Da protein peak, a 12.2 kDa protein peak, and combinations thereof, wherein the peak is determined by mass spectroscopy.

10. An isolated protein biomarker of amyotrophic lateral sclerosis selected from the group consisting of a 4367 Da protein peak, a 4660 Da protein peak, an 8547 Da a protein peak, an 8611 Da protein peak, an 8725 Da protein peak, an 8735 Da protein peak, an 8737 Da protein peak, an 8943 Da protein peak, a 9528 Da protein peak, and combinations thereof, wherein the peak is determined by mass spectroscopy.

11. An isolated protein biomarker of amyotrophic lateral sclerosis selected from the group consisting of a 2046 Da protein peak, a 3208 Da protein peak, a 4803 Da protein peak, a 5210 Da protein peak, a 5366 Da protein peak, a 6174 Da protein peak, a 6467 Da protein peak, a 7661 Da protein peak, a 8557 Da protein peak, a 9905 Da protein peak, a 10863 Da protein peak, a 12357 Da protein peak, a 14830 Da protein peak, a 14992 Da protein peak, a 15835 Da protein peak, a 16019 Da protein peak, a 16777 Da protein peak, and combinations thereof, wherein the peak is determined by mass spectroscopy.

12. The protein biomarker of claim 11, wherein the biomarker is isolated from in the spinal cord.

13. An isolated protein biomarker of amyotrophic lateral sclerosis selected from the group consisting of a 5552 Dalton (Da) protein peak, a 5960 Da protein peak, a 6187 Da protein peak, a 6260 Da protein peak, a 6274 Da protein peak, a 7093 Da protein peak, a 8754 Da protein peak, a 18044 Da protein peak, a 18257 Da protein peak, a 20930 Da protein peak, a 22885 Da protein peak, a 23400 Da protein peak, and a 23596 Da protein peak, wherein the peak is determined by mass spectroscopy.
